(19) United States
(12) Patent Application Publication
(10) Pub. No.: US 2026/0188524 A1
(43) Pub. Date: Jul. 2, 2026

(54) MEDICAL INFORMATION EXTRACTION USING A TREE OF LLM PROMPTS

(71) Applicant: GE Precision Healthcare LLC, Waukesha, WI (US)

(72) Inventors: Renato Keshet, Haifa (IL); Hrithik Auchar, Bangalore (IN); Carmen Marie Watson, Smithville, TX (US); Annamraju Ravi Bhardwaj, Bengaluru (IN); Vinoth Babu Ganesan, Bangalore (IN); Mahbubur Rahman, Bonney Lake, WA (US)

(21) Appl. No.: 19/003,720

(22) Filed: Dec. 27, 2024

(51) Int. Cl. G16H 50/70; G16H 15/00

(52) U.S. Cl. CPC G16H 50/70; G16H 15/00

(57) ABSTRACT

Techniques regarding autonomously facilitating the optimization of medical information to generate structured medical information are provided. For example, one or more embodiments described herein can comprise a system, which can comprise a memory that can store computer executable components. The system can also comprise a processor, operably coupled to the memory, and that can execute the computer executable components stored in the memory. The computer executable components can comprise a receiving component that receives Fast Healthcare Interoperability Resources (FHIR) from a FHIR server. The computer executable components can also comprise an extraction component that extracts unstructured information for medical reports by using a tree of large language model (LLM) prompts and logic for navigating the tree as a function of prompt responses. The computer executable components can also comprise a generation component that generates JavaScript Object Notation (JSON) structured data, and integrates it into original FHIR resources.

### TECHNICAL FIELD

The subject disclosure relates generally to an automated Artificial Intelligence (AI) solution for extracting unstructured information for medical reports/notes by using a tree of Large Language Model prompts, and logic for navigating the tree depending on the prompt responses and producing structured medical information.

### BACKGROUND

Healthcare professionals, particularly in oncology, generate extensive textual data daily, including doctors' notes, radiology reports, pathology reports, and consultation summaries. However, this unstructured data can pose a significant challenge for computational tools such as Clinical Decision Support (CDS) systems and patient dashboard applications, as it cannot be directly utilized.

### SUMMARY

The following presents a summary to provide a basic understanding of one or more embodiments of the invention. This summary is not intended to identify key or critical elements or delineate any scope of particular embodiments or any scope of associated claims. Its sole purpose is to present concepts in a simplified form as a prelude to the more detailed description that is presented later. In one or more embodiments described herein, devices, systems, computer-implemented methods, apparatus or computer program products that facilitate improved deep learning image processing are described.

According to an embodiment, a system is provided. The system can comprise a memory that can store computer executable components. The system can also comprise a processor that executes computer-executable components stored in a non-transitory computer-readable memory, wherein the computer-executable components can comprise a receiving component that receives Fast Healthcare Interoperability Resources (FHIR) from a FHIR server. The computer executable components can also comprise an extraction component that extracts unstructured information for medical reports by using a tree of Large Language Model (LLM) prompts and logic for navigating the tree as a function of prompt responses. The computer executable components can further comprise a generation component that generates JavaScript Object Notation (JSON) structured data, and integrates it into original FHIR resources.

According to another embodiment, a computer-implemented method is provided. The computer-implemented method can comprise receiving, by a system operatively coupled to a processor, Fast Healthcare Interoperability Resources (FHIR) from a FHIR server. Also, the computer-implemented method can comprise extracting, by a system operatively coupled to a processor, unstructured information for medical reports by using a tree of Large Language Model (LLM) prompts and logic for navigating the tree as a function of prompt responses. Also, the computer-implemented method can further comprise generating, by a system operatively coupled to a processor, JavaScript Object Notation (JSON) structured data, and integrates it into original FHIR resources.

According to a further embodiment, a computer program product that can extract unstructured information for medical reports/notes by using a tree of Large Language Model prompts, and logic for navigating the tree depending on the prompt responses and producing structured medical information is provided. The computer program product comprising a computer readable storage medium having program instructions embodied therewith, the program instructions executable by a processor to cause the processor to receive, by a system operatively coupled to a processor, Fast Healthcare Interoperability Resources (FHIR) from a FHIR server. Also, the program instructions can further cause the processor to extract, by a system operatively coupled to a processor, unstructured information for medical reports by using a tree of large language model (LLM) prompts and logic for navigating the tree as a function of prompt responses. Also, the program instructions can further cause the processor to generate, by a system operatively coupled to a processor, JavaScript Object Notation (JSON) structured data, and integrates it into original FHIR resources.

### DESCRIPTION OF THE DRAWINGS

FIG. 1 illustrates a block diagram of an example, non-limiting system that facilitates optimizing medical information in accordance with one or more embodiments described herein.

FIG. 2 illustrates an example of healthcare unstructured data in accordance with one or more embodiments described herein.

FIG. 3 illustrates a flow diagram of an example non-limiting process for the retrieval of unstructured data, optimization of unstructured, and storage of newly structured data in accordance with one or more embodiments described herein.

FIG. 4 illustrates an example, non-limiting generic prompt structure in accordance with one or more embodiments described herein.

FIG. 5 illustrates an example, non-limiting generic prompt structure populated with Oncology specific example data that provides an example of the tree of prompts in accordance with one or more embodiments described herein.

FIGS. 6 and 7 are tables containing non-limiting example a tree of prompts for extracting oncological information from the patient reports/notes in accordance with one or more embodiments described herein.

FIG. 8 is an example radiology report containing unstructured data in accordance with one or more embodiments described herein.

FIGS. 9 and 10 present an example JSON (JavaScript Object Notation) output file in accordance with one or more embodiments described herein.

FIG. 11 illustrates a block diagram of an example, non-limiting operating environment in which one or more embodiments described herein can be facilitated.

FIG. 12 illustrates an example networking environment operable to execute various implementations described herein.

### DETAILED DESCRIPTION

The following detailed description is merely illustrative and is not intended to limit embodiments or application/uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, the one or more embodiments can be practiced without these specific details.

Healthcare professionals, particularly in oncology, generate extensive textual data daily, including doctors' notes, radiology reports, pathology reports, and consultation summaries. However, this unstructured data poses a significant challenge for computational tools such as Clinical Decision Support (CDS) systems and patient dashboard applications, as it cannot be directly utilized. Natural language processing (NLP) addresses this issue by extracting meaningful information from unstructured clinical text, transforming it into structured, actionable insights.

The transformation of textual data facilitates insight extraction, enhanced decision-making, and standardization and interoperability. NLP systems can analyze free-text reports to identify critical elements like diagnoses, treatments, medications, and patient outcomes. The accurate extraction of clinical information supports evidence-based decision-making. Structured data ensures consistency across different healthcare systems and enables seamless integration.

Unstructured clinical information usually comes as part of Fast Healthcare Interoperability Resources (FHIR) resources from an Electronic Medical Record (EMR). The invention described herein is a method to extract valuable information from unstructured medical report (in particular, oncology report) and transform it into structured data formatted within FHIR standard resources using medical (in particular, oncology) structured information, using large language model (LLM) based technology. This methodology enhances and enriches existing FHIR resources by providing clinical structured interpretation of the reports.

A Large Language Model (LLM) is a type of artificial intelligence algorithm designed to understand and generate human language. These models leverage neural network techniques with extensive parameters to process and comprehend text using self-supervised learning techniques. LLMs are capable of performing a wide range of tasks such as text generation, machine translation, summarization, and even code generation.

Large language models (LLMs) are transforming medicine by enhancing data analysis, improving patient care, supporting research, and enabling more personalized treatments. By leveraging the vast amounts of structured and unstructured data in medicine, particularly oncology-such as patient records, clinical notes, research studies, and imaging reports-LLMs can help healthcare professionals gain insights, streamline workflows, and provide patients with better outcomes.

Key applications of LLMs in medicine, for example in cardiology, oncology, and neurology include clinical documentation and medical records, clinical decision support, medical research and literature review, patient monitoring and symptom tracking, precision oncology and genomic data analysis, improving access to clinical trials, enhanced patient education and engagement, and radiology and imaging analysis.

LLMs can automatically generate, summarize, and organize clinical documentation, freeing up time for medical professionals by capturing details from voice or written inputs. Data extraction is enhanced by LLMs because LLMs can pull relevant data, like cancer type, staging, and treatment history, from unstructured clinical notes and radiology reports to create structured records for clinical use. By synthesizing data from research, guidelines, and patient records, LLMs can support medical doctors such as cardiologists, oncologists, and neurologists in selecting treatments tailored to individual cases. LLMs, when trained with relevant clinical data, can help estimate prognosis, predict treatment side effects, and identify high-risk patients, supporting proactive care decisions.

Oncology research is vast and constantly evolving. LLMs can analyze thousands of publications to produce concise summaries and insights, helping clinicians and researchers stay current on new findings. By identifying patterns or emerging trends in the literature, LLMs assist researchers in generating hypotheses for further investigation, such as potential biomarkers or novel therapeutic targets.

LLMs can process patient feedback from surveys or electronic diaries, identifying symptoms, side effects, or mental health concerns for timely intervention. Remote monitoring can be achieved when LLMs analyze real-time data from patients in remote monitoring programs, spotting potential complications early and alerting clinicians for follow-up. LLMs can help interpret complex genetic data, associating genetic mutations with potential treatment pathways, particularly in cancers with genetic targets, like some breast or lung cancers. By integrating genetic and clinical data, LLMs provide insights into personalized treatment options, identifying patients who may benefit from targeted therapies or clinical trials.

LLMs can rapidly match patients with relevant clinical trials by analyzing eligibility criteria against patient data, making it easier for clinicians to identify trial options for eligible patients. Informed consent can be simplified by LLMs as they can generate understandable summaries of complex trial information, helping patients make informed choices about participating in clinical trials. LLMs can be used to provide patients with tailored information on their diagnosis, treatment options, and side effects in understandable language. Chatbots powered by LLMs can answer patient questions on symptom management and treatment side effects, encouraging adherence to treatment plans. LLMs assist radiologists by generating summaries or recommendations based on imaging results, which can then be incorporated into patient records. Combined with computer vision, LLMs can analyze textual interpretations of imaging, assisting radiologists in identifying tumor growth or response to treatment.

Using LLMs in medicine provides numerous benefits. Efficiency and time savings can be realized by automating documentation, data analysis, and literature review which frees up time for medical professionals to focus on patient care. LLMs enhance data extraction and interpretation accuracy, reducing human errors in documentation and information retrieval. LLM-powered insights help provide more accurate prognoses, personalized treatments, and timely interventions, leading to better patient outcomes. By creating accessible and structured information, LLMs facilitate collaboration among oncologists, pathologists, radiologists, and geneticists in patient management.

While highly beneficial LLMs have challenges and considerations that need to be taken into account. LLMs require strict adherence to data protection standards to ensure patient data privacy, especially given the sensitive nature of medical records. Bias in training data can lead to disparities in treatment recommendations or prognosis estimates, so models need to be validated across diverse populations. In clinical contexts, it is critical for LLMs to provide explanations for their recommendations, ensuring clinicians understand and trust the model's insights.

LLMs in oncology enhance data-driven decision-making, streamline clinical workflows, and support personalized treatments. While challenges remain, these tools have the potential to transform oncology by providing actionable insights, improving efficiency, and enabling better patient-centered care.

Deep learning has become a powerful tool in oncology, supporting cancer diagnosis, treatment planning, prognosis, and research by analyzing complex data types such as medical images, genomics, pathology reports, and clinical notes. Its capacity for pattern recognition and predictive analytics enables it to process vast datasets and identify subtle patterns that may be missed by human experts.

Deep learning models can analyze CT scans, MRIs, PET scans, and mammograms to detect tumors with high accuracy. These models are trained to recognize features associated with various cancer types, supporting radiologists in early and accurate diagnosis. Digital pathology is enhanced by deep learning models that can analyze tissue samples from biopsies, identifying cancerous cells and assessing tumor characteristics, such as cell morphology and mitotic count. Deep learning models, especially convolutional neural networks (CNNs), can outline and segment tumors in medical images, providing precise measurements of tumor size, location, and shape, which are critical for treatment planning and monitoring. Deep learning models help classify cancer types and subtypes based on imaging and histopathological features, which aids in choosing the most effective treatment approach.

Deep learning combines radiomic (imaging) data with genomic data to predict how a patient's cancer will respond to specific treatments, like chemotherapy or radiation. This helps personalize treatment plans based on individual tumor characteristics. Models trained on patient demographics, tumor data, and treatment history can help predict patient outcomes, such as survival rates or the likelihood of recurrence, providing valuable guidance for oncologists. By analyzing genetic data and understanding tumor biology, deep learning models can identify potential drug targets for specific cancer types, accelerating the drug discovery process. Deep learning models trained on large pharmacogenomic datasets predict how cancer cells respond to various drugs, which can support oncologists in selecting effective therapies and reduce trial-and-error in treatment.

Deep learning models can identify biomarkers associated with different cancer subtypes or genetic mutations, enabling more personalized treatment approaches. This is particularly valuable in cancers with known genetic markers, like breast or lung cancer. Models trained on clinical data, treatment outcomes, and medical literature can provide oncologists with treatment recommendations tailored to individual patients, incorporating factors like tumor genetics, patient health, and prior treatments.

Deep learning models can analyze patient records and identify individuals who match clinical trial eligibility criteria, accelerating the recruitment process. By identifying patients more accurately, deep learning enhances trial enrollment efficiency, helping researchers access a larger and more relevant patient population for experimental therapies.

For patients receiving treatment at home, deep learning models can analyze data from wearables, electronic health records, and patient-reported outcomes to monitor symptoms and detect early signs of complications. Models can analyze patient data for signs of potential adverse reactions to treatments, such as chemotherapy, and alert healthcare providers to intervene early, improving patient safety. Deep learning models can assist in radiation therapy planning by mapping tumors and surrounding tissues, helping to minimize damage to healthy tissue. This improves the precision and safety of radiation delivery. Deep learning models can predict the optimal radiation dose for individual patients, balancing treatment efficacy with the risk of side effects.

Furthermore, deep learning also enables the analysis of large-scale data from genomic databases, clinical trials, and cancer registries, identifying trends and generating new insights for research. By identifying patterns and relationships within complex datasets, deep learning supports oncologists and researchers in generating hypotheses, such as potential biomarkers or new treatment combinations.

Of note, in the context of this innovation, deep learning models can extract important information from unstructured data sources, such as pathology reports, oncologist notes, and medical literature, helping to build comprehensive patient profiles. NLP deep learning models can analyze social media posts, forums, and patient-reported data to gather real-world evidence on patient experiences and outcomes.

AI in oncology supports improved patient care, personalized treatment planning, early diagnosis, and cancer research by analyzing vast data sources and generating actionable insights. From diagnostic imaging to patient monitoring and drug discovery, AI has become an essential tool in oncology, empowering clinicians and researchers to enhance accuracy, efficiency, and patient outcomes.

AI, particularly deep learning models, is widely used in analyzing radiology images (CT, MRI, PET scans) to identify cancerous lesions, track tumor progression, and support early diagnosis. AI algorithms can highlight suspicious areas for radiologists, improving detection rates and potentially catching cancer in its early stages. AI systems analyze histopathological slides, identifying cancerous cells and grading tumor characteristics. This helps pathologists make accurate diagnoses by quickly highlighting malignant regions.

AI uses patient data, including genomics and prior treatment outcomes, to recommend personalized treatment plans. By integrating clinical guidelines with patient specifics, AI systems assist oncologists in selecting optimal therapies.

AI enables clinical decision support (CDS), which helps clinicians make data-driven decisions by providing insights into patient histories, cancer subtypes, and potential outcomes. This assists with selecting appropriate treatments, assessing risks, and improving overall treatment efficacy.

AI aids in the interpretation of genetic data, identifying mutations and biomarkers associated with specific cancers. This information supports precision oncology by helping clinicians tailor treatments based on genetic profiles. By analyzing genomic and proteomic data, AI can identify biomarkers that predict treatment responses or indicate prognosis, paving the way for more targeted and effective therapies.

NLP algorithms can analyze clinical notes, extracting relevant information such as cancer stage, treatment history, and symptoms. This enables clinicians to build comprehensive patient profiles and inform decision-making. NLP processes patient feedback and symptom reports, providing insights into patient experiences and enabling more personalized care plans.

AI is revolutionizing oncology by enhancing diagnostics, personalizing treatment, and accelerating research. Through its applications in medical imaging, precision oncology, patient monitoring, and drug discovery, AI is helping to improve outcomes and streamline oncology care. While challenges remain, ongoing advancements in AI promise to make cancer care more effective, personalized, and accessible.

Embodiments unlock the power of CDS tools allowing them to benefit from structured and standardized healthcare data through FHIR enriched data, which enhances and simplifies data interpretation and exploitation. Patient dashboards also benefit by providing clinical structured data, therefore, simplifying integration and interpretation of clinical reports. Unlocking CDS algorithms on patient data benefits patients directly, especially in oncology domain.

A novelty of this innovation is usage of a tree of LLM prompts for information extraction from a medical report, where the selection of each prompt is done based on an answer of a previous prompt. An embodiment of a specific tree of prompts can be of high value by being created with the guidance of domain specific healthcare experts. It is to be appreciated that other embodiments can likewise be of significant value and created automatically without user input. For example, machine-learning, artificial intelligence, quantum computing systems can be utilized.

More specifically, given a medical report or note, a first prompt is submitted to a LLM service, and its answer is used to select a second prompt, and so on. The prompt tree is pre-designed and fixed, and the process is applied without human supervision or intervention.

The tendency today regarding LLM prompt engineering is to encapsulate all the questions and cases into one single prompt. An alternative is to break a big prompt into smaller prompts and merge the results afterward. However, in this alternative all the small prompts are used in all cases, and there is not selection of prompts based on the answer of previous prompt.

A concept called Tree of Thoughts applied to LLM prompts, does not target information extraction from medical reports, but rather seeks to solve complex problems by dealing with them in small steps mimicking the human process of reasoning. It uses the intermediate steps to generate thoughts, which guide the resolutions of the problem. This is different from our invention, which does not generate intermediate thoughts, but rather applies pre-designed logic for selecting the best questions for a given text. Embodiments described herein generate structured data from unstructured data using tree of prompts. Advantages are that a standardized data format is generated that can be readily utilized by servers in a streamlined and highly efficient manner and be understood without undergoing tedious review of unstructured data as in conventional systems. Thus, processing speed, memory utilization (e.g., indexing, write/read efficiency), energy consumption are vastly improved as compared to conventional systems.

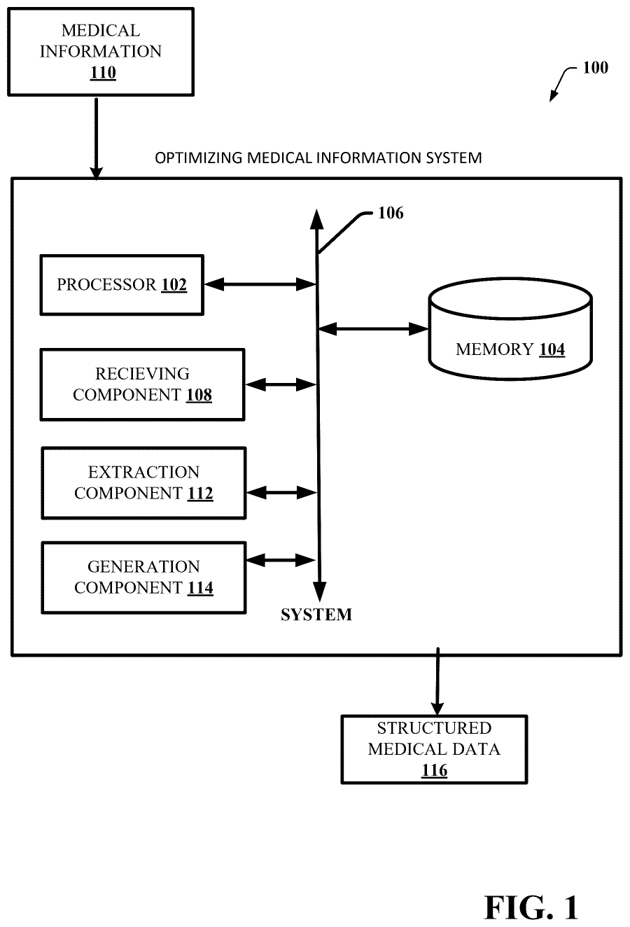

FIG. 1 illustrates a block diagram of an example, non-limiting system that facilitates optimizing medical information in accordance with one or more embodiments described herein. As shown, an optimizing medical information system 100 can be electronically integrated, via any suitable wired or wireless electronic connection, with medical information 110. In various embodiments, the medical information 110 can depict any suitable anatomical structure of any suitable medical patient. As some non-limiting examples, the medical information could be patient demographic information; clinical data such as diagnosis, medications, and treatment plans, lab results, vital signs, imaging results, and progress notes; medical history; procedural data such as records of surgeries, biopsies, or other medical procedures, anesthesia reports and surgical notes; behavioral and lifestyle data such as smoking, alcohol consumption, or drug use, exercise routines and diet, sleep patterns and mental health evaluations; administrative and billing information; genomic and molecular data such as DNA sequencing information and biomarker analyses; real-time health data such as data from wearable devices like fitness trackers or medical implants and remote patient monitoring systems.

In various embodiments, the medical information optimizing system 100 can comprise a processor 102 (e.g., computer processing unit, microprocessor) and a non-transitory computer-readable memory 104 that is operatively or communicatively connected or coupled to the processor 102. The non-transitory computer-readable memory 104 can store computer-executable instructions which, upon execution by the processor 102, can cause the processor 102 or other components of the medical information optimizing system 100 (e.g., receiving component 108, extraction component 112, generation component 114) to perform one or more acts. The system 100 can further comprise a system bus 106 that can couple to various components such as, but not limited to, receiving component 108, extraction component 112, generation component 114, memory 104 and/or a processor 102. In various embodiments, the non-transitory computer-readable memory 104 can store computer-executable components (e.g., receiving component 108, extraction component 112, generation component 114), and the processor 102 can execute the computer-executable components.

In various embodiments, the medical information optimizing system 100 can comprise a receiving component 108. In various aspects, the receiving component 108 can electronically receive a set of medical information 110. In various instances, the receiving component 108 can electronically retrieve the medical information 110 from any suitable centralized or decentralized data structures (not shown) or from any suitable centralized or decentralized computing devices (not shown). As a non-limiting example, whatever medical device, equipment, or modality (e.g., Fast Healthcare Interoperability Resources (FHIR) from a FHIR server) that generated or captured the medical information 110 can transmit the medical information 110 to the receiving component 108. In any case, the receiving component 108 can electronically obtain or access the medical information 110, such that other components of the medical information optimizing system 100 can electronically interact with the medical information 110.

Various embodiments described herein can be considered as a computerized tool (e.g., any suitable combination of computer-executable hardware or computer-executable software) that can facilitate improved deep learning medical information processing. In various aspects, such a computerized tool can comprise a receiving component 108, an extraction component 112, or a generation component 114.

In various embodiments, an extraction component 112 of the system can extract unstructured information from medical reports (medical information 110) by using a tree of large language model (LLM) prompts and logic for navigating the tree as a function of prompt responses. Generative artificial intelligence (AI) is a type of AI that uses machine learning to create new content, such as text, images, music, audio, and videos. Generative AI models are trained on large amounts of data, and then use that data to learn how to create new content that's statistically likely to be relevant to a given prompt. For example, a generative AI model trained on text can respond to written prompts in a way that seems organic and original. As more and more prompt engineering is used for LLM based AI, there is a tendency to design one big prompt that is general enough to get the desired responses for any case. The usage of a single prompt for complex tasks conveys the risk of the prompt not being appropriate for particular situations, which can cause hallucinations or other mistaken responses from the LLM. By breaking the prompts in a tree of prompts, and applying each prompt to the corresponding situation, the risk is potentially reduced in a significant way, which leads to higher accuracy and reliability from the LLM-based system.

In any case, a tree of prompts can be configured, as described herein, to receive as input medical information and to produce structured medical information. Accordingly, the generation component can electronically execute the deep learning neural network on the medical image, thereby yielding a set of region-wise parameter maps corresponding to the medical image. More specifically, the extraction component 112 can feed the medical information 110 to the generation component 114 an input layer of the tree of prompts, the medical information can complete a forward pass through one or more prompts, and an output layer of the can generate structured medical information based on activations generated by the one or more hidden layers.

In various embodiments, a generation component 114 of the computerized system 100 can electronically generate JavaScript Object Notation (JSON) structured data, on any suitable electronic medium. The newly generated JSON structured data can then be integrated into the original FHIR resources. Thus, a user, technician, medical professional, or subject matter expert can visually inspect or view the JSON structured data. The data can then be sent, in its structured form to be integrated into the original FHIR resources which can aid the user, technician, or medical professional in making a diagnosis or prognosis. Take an example of generic cancer data extraction vs prostate cancer vs breast cancer specific data extraction. A uro-oncologist for prostate cancer is expecting to see the terminology related to prostate cancer for example Gleason score which describes the aggressiveness of prostate cancer. A breast oncologist is expecting to see Nottingham score which is a grading system for breast cancer that assesses how abnormal breast cancer cells look and grow compared to normal cells. Both of these scores fall under a generic umbrella of histology grade. Similarly, breast and prostate cancers involve additional hormonal treatment choices beyond generic treatments. The prompting structure allows the posing of the correct prompt(s) as applicable exposing placeholders under each generic prompt. This specific cancer type data yields valuable information which can also aid in medical decisions, for example making a diagnosis or prognosis.

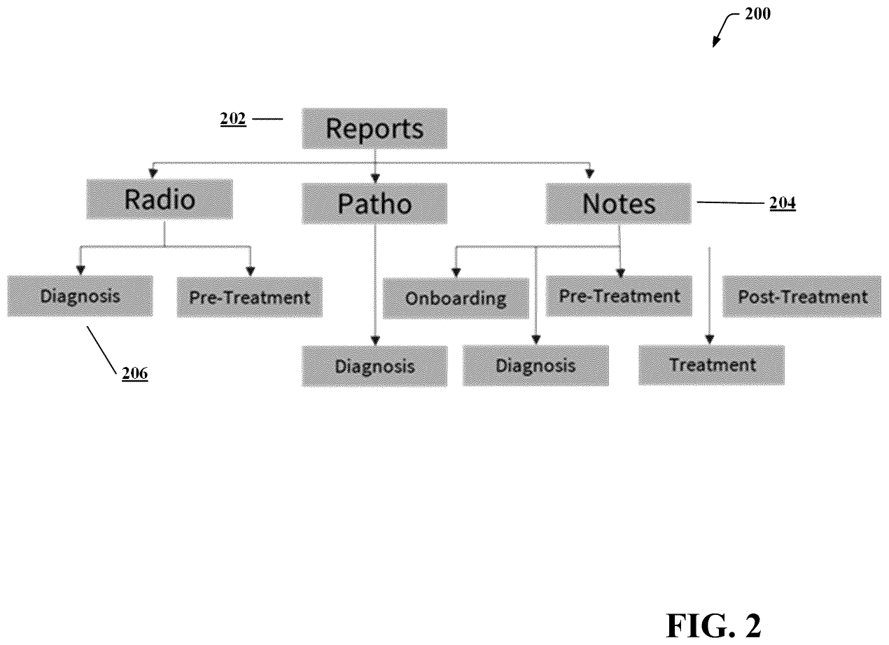

FIG. 2 illustrates a non-limiting example of healthcare unstructured data in accordance with one or more embodiments described herein. 202 Reports refers to healthcare textual data. Reports in modern healthcare are essential for personalizing treatment, ensuring accuracy and continuity, and facilitating collaboration among healthcare providers. In healthcare, reports allow for ongoing assessment and adaptation, making them a cornerstone in delivering high-quality medical care.

Reports can be sub-categorized by treatment type 204. In this non-limiting example, the treatment types are Radio (Radiology), Patho (Pathology) and Notes. In the context of oncology reports document diagnosis and cancer type. Pathology reports document the initial diagnosis, including cancer type, stage, grade, and molecular characteristics, which guide treatment decisions. Specific cancer types (e.g., carcinomas, sarcomas) and genetic markers (e.g., HER2, BRCA mutations) are documented, enabling targeted treatment choices. Oncology reports document each stage of treatment, from chemotherapy cycles to radiation therapy sessions, allowing providers to assess effectiveness. Reports track tumor size changes, symptoms, and lab results to determine how well the treatment is working and adjust as needed.

The treatment phase 206 provides additional classification data on the treatment type 204. The treatment phases can vary based on treatment type as seen in FIG. 2, this can be a one-to-many relationship. Radio is comprised of Diagnosis and Pre-Treatment. In contrast Patho has only one phase, Diagnosis. If a tumor progresses or side effects worsen, oncology reports help guide treatment changes, such as switching to a different drug, adjusting dosage, or adding supportive therapies. Oncology treatments often have significant side effects. Reports help track these in detail, enabling timely interventions and adjustments. Accurate, standardized reports provide data essential for clinical trials and research on new oncology treatments. Based on documented characteristics like molecular markers, reports can help match patients to suitable clinical trials or experimental therapies. For further comparison, Notes has 5 categories in this non-limiting example. Detailed reports support accurate billing, authorization, and reimbursement for treatments and procedures. Reports serve as formal records of the patient's treatment and progression, which is important in medical-legal cases or for patient rights. Summarized reports can help explain complex treatments, potential outcomes, and side effects to patients, fostering informed decision-making. Consistent reporting allows patients to see their progress, helping them stay engaged and motivated throughout their treatment journey.

FIG. 2 illustrates a method of medical report classification that lends itself to a novelty of this invention that utilizes top-down hierarchical prompting type by which the first prompt is designed to extract the treatment type and the phase automatically. Refinement prompts can be subsequently implemented to target specific subsets of the extracted entities to refine or clarify them. In Natural Language Processing (NLP), based on the treatment phase and the type of report, the entity extractions are defined. Fine tuning prompts can then be used to address ambiguities or conflicts in the extracted data. This adds fine tuning to the core specialty, for example oncology and cancer type.

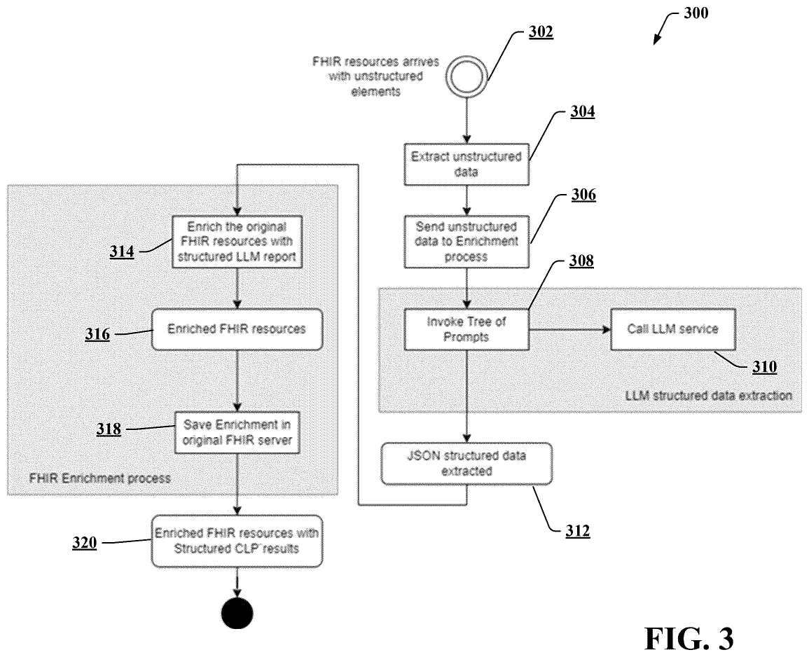

FIG. 3 illustrates a flow diagram of an example non-limiting process for the retrieval of unstructured data, optimization of unstructured, and storage of newly structured data in accordance with one or more embodiments described herein. Unstructured clinical information usually comes as part of Fast Healthcare Interoperability Resources (FHIR) resources from an Electronic Medical Record (EMR). In the context of FHIR, refers to the digital version of a patient's health record that is used within a single healthcare organization, such as a clinic or hospital. EMRs contain a range of patient information, including medical history, diagnoses, treatment plans, immunizations, allergies, lab results, and more. FHIR helps make EMRs more interoperable, allowing them to share data seamlessly with other systems or applications beyond the individual healthcare organization.

For this innovation, the basic process starts at 302 with the arrival of Fast Healthcare Interoperability Resources (FHIR) with unstructured elements. FHIR is a standard developed by the Health Level Seven International (HL7) organization. It's designed to facilitate the electronic exchange of healthcare information between different systems in a fast, standardized, and secure manner. FHIR uses modern web technologies like RESTful APIs, JSON (JavaScript Object Notation), and XML, which make it accessible and compatible with many contemporary web-based systems.

FHIR facilitates the integration of data from multiple providers and sources (e.g., lab results, medications, past medical history), giving clinicians a comprehensive view of the patient. Furthermore, FHIR enables patients to access their health information easily through apps and portals, empowering them to be more involved in their own care and make informed decisions. The modular design of FHIR and lightweight framework make it faster than previous interoperability standards. Real-time data exchange is essential in emergency care, telemedicine, and time-sensitive treatments. With real-time access to patient data, healthcare providers can make more accurate, timely decisions, improving patient outcomes.

FHIR's compatibility with modern web technologies encourages the development of innovative healthcare apps, tools, and devices. With FHIR, developers can build custom apps tailored to specific health needs (e.g., chronic disease management, remote monitoring), driving advancements in personalized medicine. FHIR makes it easier to collect standardized data across large populations, supporting clinical trials, population health studies, and predictive analytics. By making healthcare data more accessible, FHIR enables health systems to analyze patterns, optimize care delivery, and improve population health.

FHIR can be configured to comply with regulations like HIPAA in the U.S., which require strict controls on access and sharing of patient information. As a result, FHIR has been adopted by regulatory bodies, including the U.S. Office of the National Coordinator for Health IT (ONC), as part of interoperability mandates, supporting standardized data access and transparency.

FHIR allows seamless sharing of data between different healthcare providers, supporting continuity of care when patients move between facilities or specialists. By providing shared access to accurate and up-to-date patient data, FHIR enables collaboration across various care teams, from primary care to specialty treatment. FHIR's use of standard internet technologies (e.g., JSON, HTTP) reduces the cost and complexity of integrating systems, particularly when compared to older standards like HL7 v2 or CDA. Healthcare organizations can adopt FHIR incrementally, allowing them to scale interoperability efforts based on their needs and resources, which reduces upfront costs. FHIR is essential for modern healthcare interoperability, enabling seamless data sharing across systems, improving patient care, supporting compliance, and fostering health technology innovation. By bridging the gap between disparate systems, FHIR helps create a more connected, accessible, and patient-centered healthcare ecosystem.

Following the reception of FHIR resources, unstructured data like the diagnostic report notes, or the clinical notes as textual data is extracted. These data are pushed to the enrichment process 306 pipeline depicted in FIG. 3.

Large Language Model (LLM) data extraction occurs by invoking the Tree of Prompts 308 which utilizes LLM service 310. LLM are advanced machine learning models trained on massive datasets, designed to understand, generate, and manipulate human language. LLMs, such as GPT-4, BERT, and T5, have a large number of parameters (often billions) and are trained on diverse text sources to perform various language-related tasks, including answering questions, summarizing text, translating languages, and more. Key features of LLMs can include Natural Language Understanding (NLU) which means that LLMs can understand the context, sentiment, and intent of a given text; they can generate coherent and contextually relevant text; many LLMs can be fine-tuned and adapt for specific domains, such as medical or legal language, to perform specialized tasks; and LLMs can often perform tasks with little to no specific task training by leveraging pre-existing knowledge. Due to their versatility, LLMs are used in a variety of applications, from chatbots to recommendation systems, content creation, and customer support. The result coming from the LLM service is a JSON file containing a list of entities, JSON structured data that has been extracted 312. The LLM model is prompted to find these entities and fill the content of found elements. These elements are related to multiples classes: Pathology, Radiology, and Notes.

The extracted entities are then normalized as a JSON structure(s), and integrated into the original FHIR resources as extension elements as part of the FHIR Enrichment Process (314, 316, 318). The added element is a JSON structure, allowing a reading application to interpret programmatically the LLM extracted results. The final enriched FHIR resources containing structured Natural Language Processing (NLP) results are pushed to the FHIR server 320. The final enriched FHIR resources residing on the FHIR server promote healthcare interoperability and data standardization. Structured output allows for the application of standard FHIR queries to directly interrogate the outcomes of the NLP. By transforming LLM results into structured formats, the system not only enhances data utility but also enriches the original datasets, unlocking deeper insights and enabling more effective data integration and analysis. Thus, medical professionals and systems have access to the final enriched FHIR resources which is a vast improvement compared to conventional systems and processes. For example, in cancer treatment there is a generic umbrella of histology grade, the final enriched FHIR resources residing on the FHIR server contain structured data containing Gleason score for prostate cancer and Nottingham score for breast cancer instead of the generic histology grade. Similarly, treatment of prostate and breast cancers involve additional hormonal treatment choices beyond generic treatments.

Natural Language Processing (NLP) focuses on analyzing and extracting valuable information from clinical and healthcare-related text. NLP is used to convert unstructured clinical notes, reports, and other healthcare documents into structured, actionable data. This process helps healthcare organizations and researchers gain insights from vast amounts of medical text data and aids in improving patient care, research, and operational efficiency. Unstructured data poses a significant challenge for computational tools such as Clinical Decision Support (CDS) systems and patient dashboard applications, as it cannot be directly utilized. NLP addresses this issue by extracting meaningful information from unstructured clinical text, transforming it into structured, actionable insights. This transformation facilitates insight extraction as NLP systems can analyze free-text reports to identify critical elements like diagnoses, treatments, medications, and patient outcomes; enhanced decision-making due to the accurate extraction of clinical information supports evidence-based decision-making and standardization and interoperability because structured data ensures consistency across different healthcare systems and enables seamless integration.

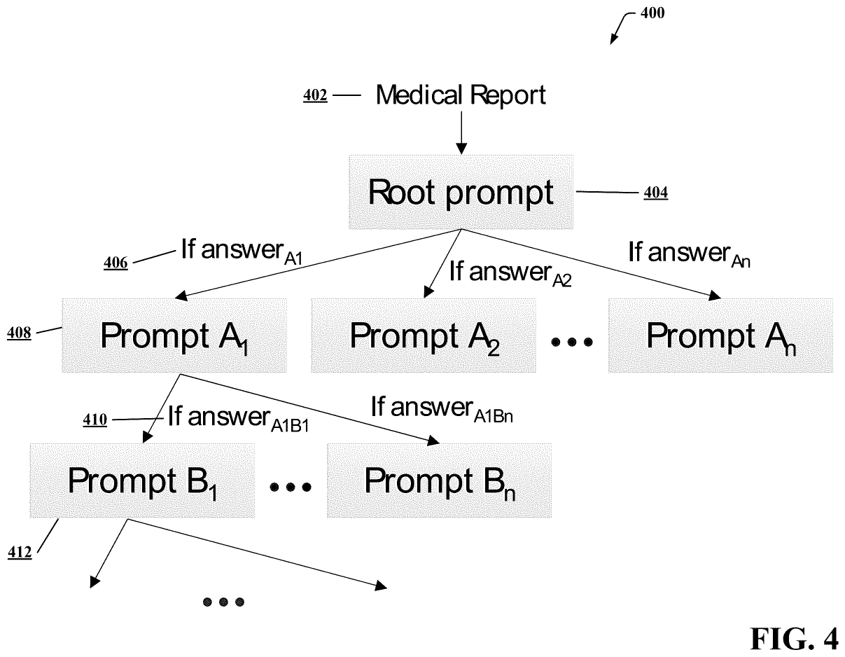

FIG. 4 illustrates an example, non-limiting generic prompt structure in accordance with one or more embodiments described herein. A generic prompt structure provides a flexible format for crafting prompts that can guide AI models effectively toward specific responses or outputs. By creating a structured approach, the prompt can help define the task, context, format, and any specific instructions. This structure can be applied across different use cases, whether generating creative content, retrieving information, or performing technical tasks. The components of a generic prompt structure include the context or background information in order to give context to the request. This helps the model understand the subject or scenario by providing any necessary background information to give context to the request. The medical report 402 provides this context in regard to the embodiments described herein. We next examine the task or instruction that is a component of the generic prompt structure 404. The purpose of this task or instruction is to clearly define what the AI needs to do and outlines any specific instructions or constraints. Further components of a generic prompt structure include any specific instructions or constraints, the preferred structure for the response. The final components of the prompt are optional such as an example which can be especially helpful for complex or ambiguous tasks and any further limitations or specifications, such as “use simpler language” or “focus on the last five years of research.” This helps narrow down the output to be more aligned with your needs. This leads to design a tree of prompts, where the answer to one prompt is used to select the next prompt from one level down. Based on the results of the root prompt, answerA1 . . . answerAN 406, further prompts (PromptA1 . . . . PromptAN) 408 are submitted specific to the answer received. This process is followed in further iterations as indicated in 410 and 412 and continues until the prompts and answers are exhausted based on the use case and criteria of the prompt structure. The ultimate goal of the process is to extract information from a given medical report by dynamically expanding the prompt tree based on switches such as cancer type or patient state.

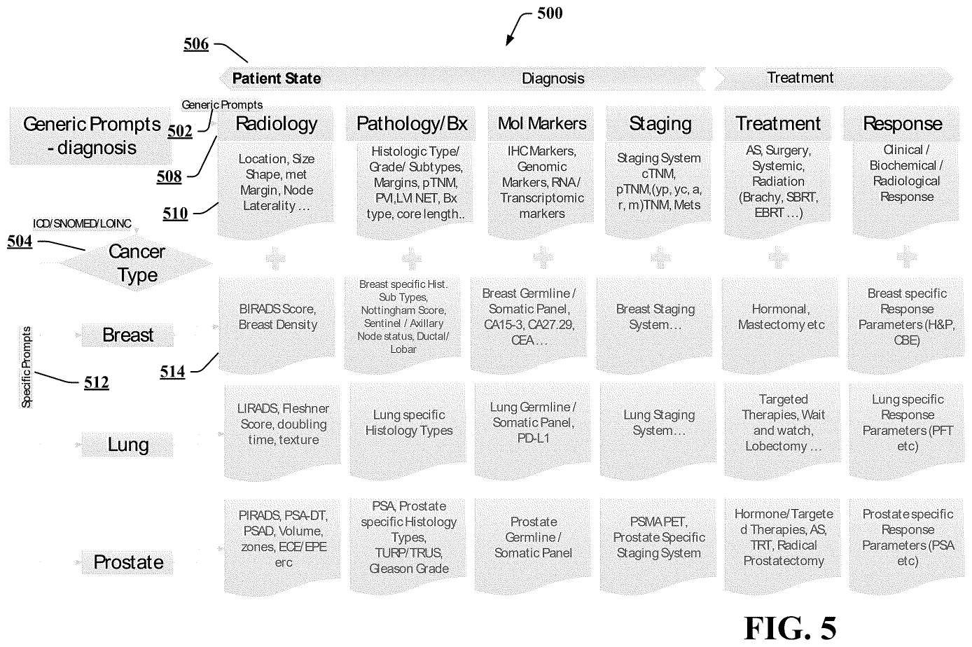

Turning to FIG. 5, illustrated is an example, non-limiting generic prompt structure populated with Oncology specific example data that provides an example of the tree of prompts defined by the invention in accordance with one or more embodiments described herein. Similar to FIG. 4 illustrated is the design for a tree of prompts, where the answer to one prompt is used to select the next prompt from one level down. The ultimate goal of the process is to extract information from a given medical report 502 by dynamically expanding the prompt tree based on switches such as cancer type 504 or patient state 506.

A medical report 502 is a detailed document that records a patient's health status, medical history, diagnoses, treatments, and other clinical information gathered during healthcare visits. It serves as a critical communication tool between healthcare providers, supports continuity of care, and is used for medical documentation, legal, and administrative purposes. A medical report is typically comprised of patient information including identifying details such as name, date of birth, sex, patient ID, and contact information; the patient's medical history which can include chronic illnesses, surgeries, allergies, previous treatments, and hospitalizations; the patients family history which can include medical conditions within the patient's family that might have genetic implications (e.g., diabetes, heart disease); the patient's social history which details lifestyle factors like smoking, alcohol consumption, occupation, and exercise habits. A medical report will further contain the primary reason for the patient's visit, often presented as the main symptom or concern (e.g., “persistent cough” or “chest pain”); the history of the present illness (HPI) which is a detailed description of the current medical issue, including onset, duration, location, severity, and any aggravating or relieving factors; the observations and findings from the doctor's physical examination, including vital signs (e.g., temperature, blood pressure, heart rate), and assessment of body systems (e.g., respiratory, cardiovascular, gastrointestinal). Where applicable a medical report can contain diagnostic test and results, for example laboratory tests (e.g., blood tests, urinalysis) and imaging studies (e.g., X-rays, CT scans) along with their results, interpretations, and findings relevant to the patient's condition; diagnosis which is a formal identification of the patient's condition based on clinical findings, symptoms, and test results. Diagnoses may include ICD codes for standardization and billing; the treatment plan which is a detailed outline of the proposed treatment, including medications, therapies, surgeries, lifestyle modifications, or follow-up appointments; current medications, dosage, and frequency. It may also include any prescribed new medications or changes to existing prescriptions; recommendations and follow-up which can include instructions on what the patient should do next, such as lifestyle changes, additional tests, or referrals to specialists. This section also includes follow-up appointment details; physician's notes, signature and date which can include additional observations, insights, or concerns noted by the healthcare provider, often providing a more subjective assessment of the patient's status along with the physician's or provider's signature and the date the report was completed, adding authenticity and accountability.

Medical reports can fall into various categories, including but not limited to, a consultation report which is a summary of findings from a specialist consultation, including diagnosis and recommended treatments; an operative report which is a detailed report of a surgical procedure, including pre- and post-operative diagnoses, surgical steps, and outcomes; a discharge summary which is provided at the end of a hospital stay, outlining the patient's status, treatments received, and instructions for aftercare; a radiology report which is a summary of imaging results, interpretations, and implications for diagnosis or treatment; a pathology which can include findings from tissue or biopsy samples, often used to confirm diagnoses like cancer; and progress notes including ongoing documentation of the patient's progress over time, used to track changes in condition or response to treatment.

Medical reports can serve a variety of purposes and uses, they are a communication took that serves as a record to inform other healthcare providers involved in a patient's care; it provides legal documentation of the patient's medical history and treatments for potential legal or insurance purposes; medical reports provide necessary documentation for coding and billing purposes in healthcare reimbursement; this tool facilitates consistent and coordinated care across different providers and care settings; and medical reports can aid in research and quality improvement when anonymized data from medical reports is used to analyze healthcare trends, improve treatment protocols, and conduct medical research.

Medical reports play a vital role in healthcare, as they ensure accurate and timely recording of patient information, guide clinical decisions, support patient safety, and help ensure quality care delivery. For patients, these records are essential in understanding their health history and managing long-term health outcomes.

Patient state 506 in oncology refers to the current clinical and overall health condition of a patient with cancer, including physical, emotional, and functional status. It encompasses vital signs, disease progression, symptom severity, treatment side effects, and mental well-being. It is a comprehensive assessment that includes the patient's physical, mental, and emotional status, as well as any underlying medical conditions and symptoms they may be experiencing. The patient's state is assessed frequently, as it directly impacts treatment decisions, adjustments, and prognosis. Understanding a patient's state 506 helps healthcare providers make informed decisions about treatment, monitor progress, and adjust care plans as necessary.

The components of a patient's state in oncology can include, the status of the disease such as tumor size, spread (metastasis), and stage of cancer. Changes in these indicators often prompt re-evaluation of treatment plans; symptoms related to cancer (e.g., pain, fatigue, weight loss) and side effects from treatments (e.g., nausea from chemotherapy) affect the patient's comfort, function, and treatment adherence; the patient's vital signs and physical condition; mental and emotional well-being as psychological aspects such as anxiety, depression, and mental resilience play a role in how well a patient can handle treatment and recovery; a patient's ability to perform daily activities (Activities of Daily Living, or ADLs) reflects the impact of both cancer and treatment. Assessments of quality of life help guide supportive and palliative care options; and blood tests and tumor markers (e.g., PSA, CA-125) provide insights into disease activity, treatment effectiveness, and potential complications.

A patient's state is significant in oncology as it is used to guide treatment decisions, monitor responses to treatment, manage side effects and symptoms, adjust treatment plans, and improve quality of life and palliative care and to access the patient's prognosis. Oncologists often adjust treatment regimens based on changes in the patient's state, such as transitioning to palliative care if curative treatments are no longer effective. The patient's state helps estimate survival rates, recovery likelihood, and potential complications, aiding in both treatment planning and patient counseling. In oncology, a patient's state is a comprehensive measure of health that influences treatment, response monitoring, and quality of life considerations. Regular assessment is essential for optimizing care, managing symptoms, and supporting informed decision-making for both patients and providers.

International Classification of Diseases (ICD), Systematized Nomenclature of Medicine-Clinical Terms (SNOMED), and Logical Observation Identifiers Names and Codes (LOINC) are standardized clinical coding systems used in healthcare to ensure consistency, interoperability, and clarity in medical documentation, billing, and research. Each serves a different purpose and has unique features, although they can be used together in electronic health records (EHRs) and clinical information systems.

Cancer type 504 refers to the specific classification of cancer based on the location, cell type, or tissue from which it originates. Classifying cancers by type helps healthcare providers choose the most effective treatments, understand how the disease may progress, and determine the likely prognosis. Cancer types 504 are generally named after the organ or tissue where the cancer begins and sometimes by the type of cell involved. Examples of cancer type include but are not limited to breast, lung, prostate.

Healthcare professionals, particularly in oncology, generate extensive textual data daily, including but not limited to doctors' notes, radiology reports, pathology reports, and consultation summaries. However, this unstructured data poses a significant challenge for computational tools such as Clinical Decision Support (CDS) systems and patient dashboard applications, as it cannot be directly utilized. Natural language processing (NLP), addresses this issue by extracting meaningful information from unstructured clinical text, transforming it into structured, actionable insights.

For different combinations of cancer type, patient state (dx/tx) 506, report type 508 and content 510, the information can be best extracted by switching to a priori set of targeted prompts. However, we assume a use case where a human cannot design or select such a prompt in real time, as the process should be completely automatic. Until cancer type 504 is determined, generic prompts 502 are used. The leaf prompts 512 in the tree should ask for specific information from the report, whereas the specific leaf prompt 514 is selected by navigating the tree of prompts, where the prompt for each level is selected automatically based on the answer of the previous prompt. This selection logic is pre-designed and fixed.

Examining a specific non-limiting example in FIG. 5 we start with a generic prompt 502, for the root prompt it is determined that the report type is Radiology 508 whose content 510 can include Location, Size, Shape, met Margin, Node Laterality, etc. Prompts of this nature continue to extract information until cancer type 504 is determined for example, Breast cancer. The specific leaf prompts 514 are automatically navigated on report type, for example Radiology, and example cancer type breast following the prompts and answers to the pertinent data, in this case data could include BIRADS Score and Breast Density. Similarly, the tree of prompts can be navigated for other report types such as Pathology/Bx, Mol Markers, Staging, Treatment, and Response and other cancer types such as lung and prostate which would navigate different paths as indicated by the prompts in the tree of prompts and the answers to these prompts which direct to the subsequent prompt to be evaluated, thus following generic prompt structure noted in FIG. 4.

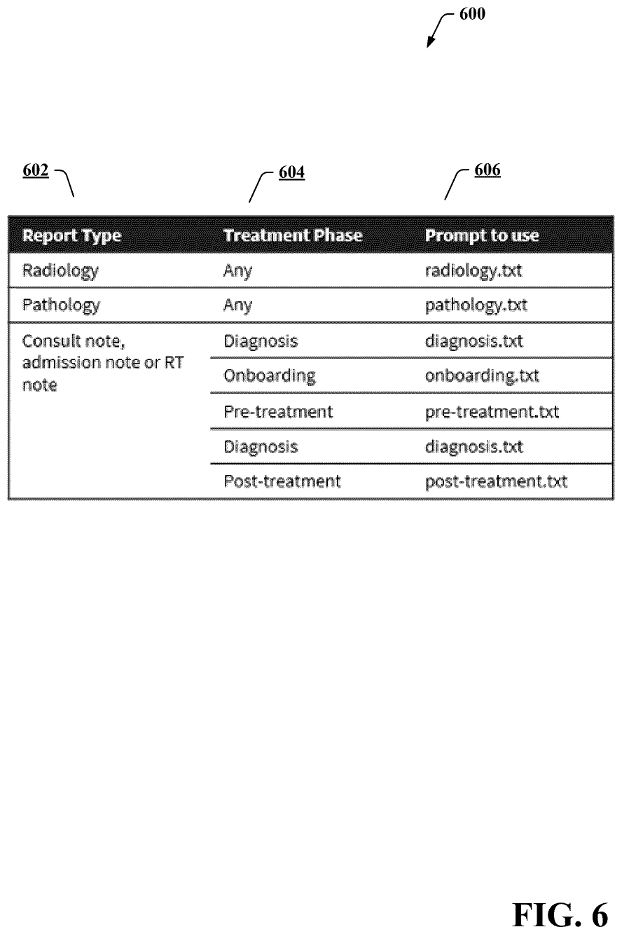

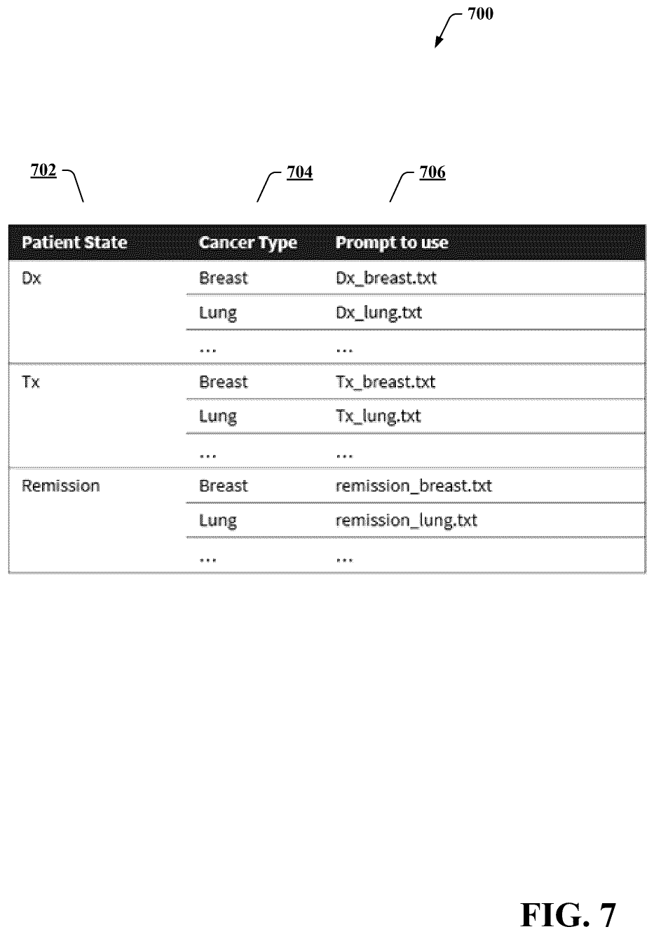

FIGS. 6 and 7 are tables containing non-limiting example a tree of prompts for extracting oncological information from the patient reports/notes in accordance with one or more embodiments described herein. A specific embodiment of this invention has been implemented in the context of a NLP component. This embodiment consists of a tree of prompts for extracting oncological information from the patient reports/notes found in the Data Fabric. Beginning at level 1, assuming this is an oncological patient, the first prompt reads (excerpt): 1. Identify the report type 602, which could be one of the following: “radiology”, “pathology”, “consult note”, “admission note”, or “RT note”. If unable to determine the type, return null. 2. Identify the treatment phase 604, which could be one of the following: “onboarding”, “pre-treatment”, “diagnosis”, “treatment”, or “post-treatment”. If unable to detect the treatment phase, return null. Moving to level 2: Depending on the answers to the level 1 questions, a prompt is selected according to the table 606. FIG. 7 details level 3, given patient state 702 and cancer type 704 obtained in level 2, a third prompt could be selected according to the table 706.

As an example, for understanding the differences between the prompts listed in the level 2 table in FIG. 6, consider the excerpts from two example prompts. An excerpt from patient onboarding using prompts defined in onboarding.txt would extract the following details if available: 1. Past Medical History: ( . . . ), 2. Patient Complaints: ( . . . ), 3. Comorbidities: ( . . . ) and 4. Performance Status: ( . . . ). Another example excerpt that utilizes patient data after a treatment would utilize prompts as defined in post-treatment.txt to extract the following details if available: 1. Tumors Information: ( . . . ), 2. Lymph Node Details: ( . . . ), and Treatment Details: ( . . . ). Notice how the requested pieces of information are targeted to the specific context discovered in level 1. The prompts are previously crafted, following subject matter expert guidance. The novelty is the usage of a tree of LLM prompts for information extraction from a medical report, where the selection of each prompt is done based on the answer of the previous prompt.

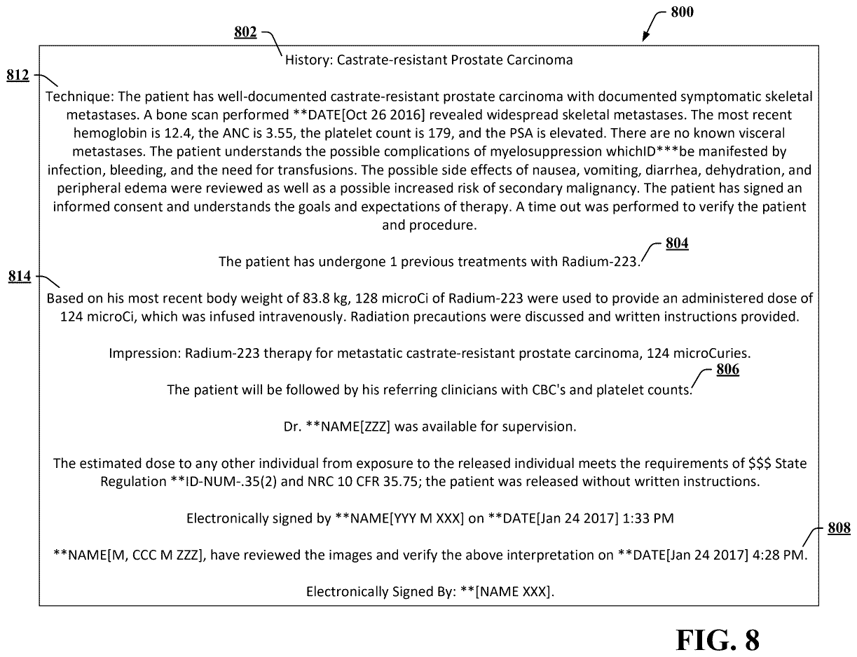

FIG. 8 is an example radiology report containing unstructured data in accordance with one or more embodiments described herein. Unstructured data of various types can be observed in the report, 802 and 804 could be components of the patient's medical history; 806 shows follow up recommendations; 808 and the subsequent line with text contain signature and date for the report. Additional unstructured data contained in the report. 812 and 814 present unstructured data specific to this radiology report that appear highly pertinent for oncology and would benefit from being structured and available for ingestion into CDS or other systems utilizing FHIR resources. This could lead directly to improved patient care and outcomes.

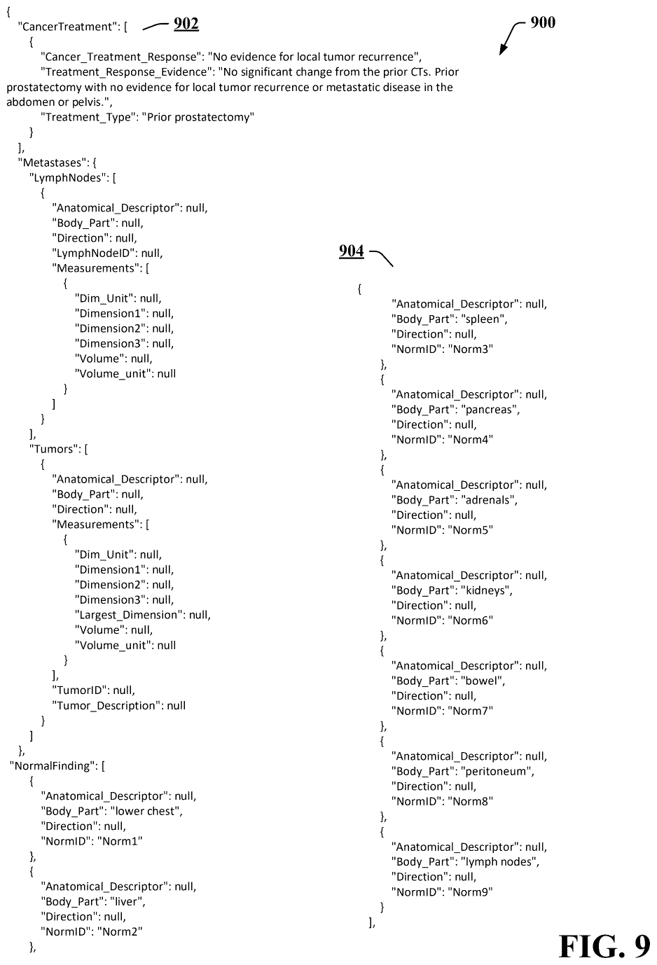

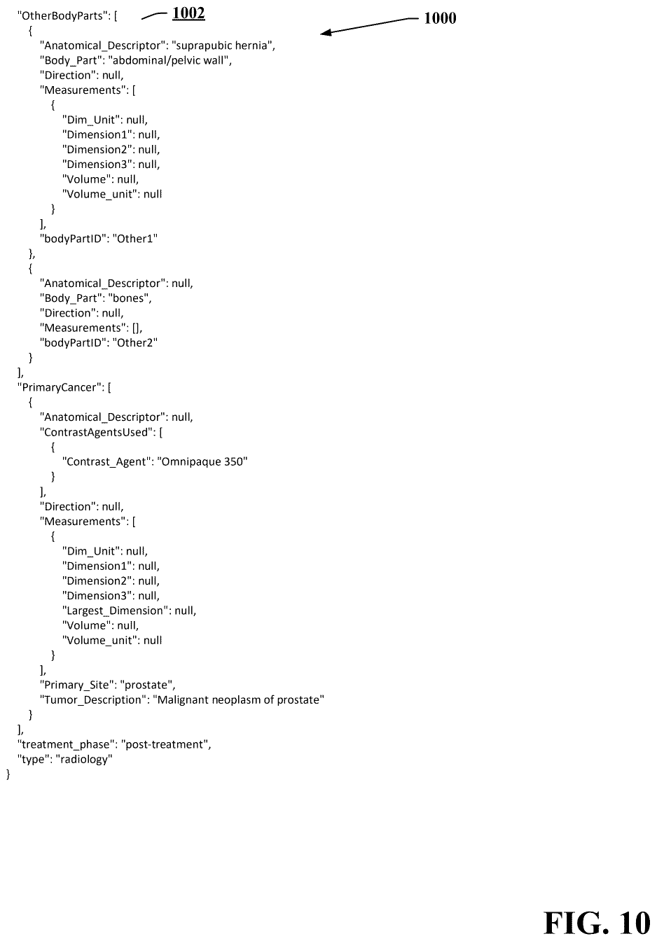

FIGS. 9 and 10 present an example JSON output file in accordance with one or more embodiments described herein. A JSON (JavaScript Object Notation) file is a lightweight data-interchange format that is easy for both humans to read and write and for machines to parse and generate. JSON uses a simple text format that stores data as key-value pairs, arrays, and nested structures, making it highly flexible for representing complex data. JSON files have a “json” file extension and are widely used for data exchange between servers and web applications, configuration files, and APIs.

A JSON file is structured with key-value pairs where keys (often strings) are mapped to values, like {“name”: “Alice”}; arrays which are ordered lists of items, like {“fruits”: [“apple”, “banana”, “cherry”]}; and nested objects which are objects can contain other objects, allowing for hierarchical data, like {“person”: {“name”: “Alice”, “age”: 30}}. JSON's minimal syntax allows it to represent complex data structures concisely, reducing data size and improving network efficiency, especially for web and mobile applications. JSON is universally supported and can be parsed by almost any programming language (Python, JavaScript, Java, etc.), making it ideal for cross-platform data exchange. JSON supports nesting and arrays, making it suitable for representing complex, hierarchical data structures. This flexibility allows it to model various data types, from simple lists to detailed configurations.

JSON is the standard format for REST APIs, making it essential for building, consuming, and integrating with web services and applications. JSON is easily parsed by machines and JSON parsing libraries are widely available, so applications can read and process JSON data with minimal code, making JSON a robust choice for machine-readable configurations and data serialization. Data validation and schema definitions are support in JSON (e.g., JSON Schema) that allow for data validation, ensuring that incoming data adheres to expected structures and content types.

JSON's readable format makes it perfect for configuration files in applications, allowing easy setup, customization, and reading of configurations. JSON files are lightweight, flexible, and universally compatible, making them ideal for data exchange in web applications, APIs, and configuration files. JSON's readability and ease of use support both human and machine interactions, offering an efficient, reliable data format across many domains.

The generated JSON file is based on knowledge of the oncology domain, and describes oncology findings, conditions, etc. The JSON is generated by the LLM service upon indication through the sent prompts. The original FHIR resources is enriched with the JSON generated, by adding an extension element to the FHIR resource. The extension element contains the full result of NLP, as well as contextual information: the version of the tool, the generation date, etc. An example of the extension added to FHIR could be
{“url”: “content[0].attachment.data”, “valueString”: “<Report Output.”}

A complete example of the generation created from the input radiology text in FIG. 8 is available in FIGS. 9 and 10; starting at 902, proceeding to 904 and completing at 1002.

The herein disclosure describes non-limiting examples. For ease of description or explanation, various portions of the herein disclosure utilize the term “each,” “every,” or “all” when discussing various examples. Such usages of the term “each,” “every,” or “all” are non-limiting. In other words, when the herein disclosure provides a description that is applied to “each,” “every,” or “all” of some particular object or component, it should be understood that this is a non-limiting example, and it should be further understood that, in various other examples, it can be the case that such description applies to fewer than “each,” “every,” or “all” of that particular object or component.

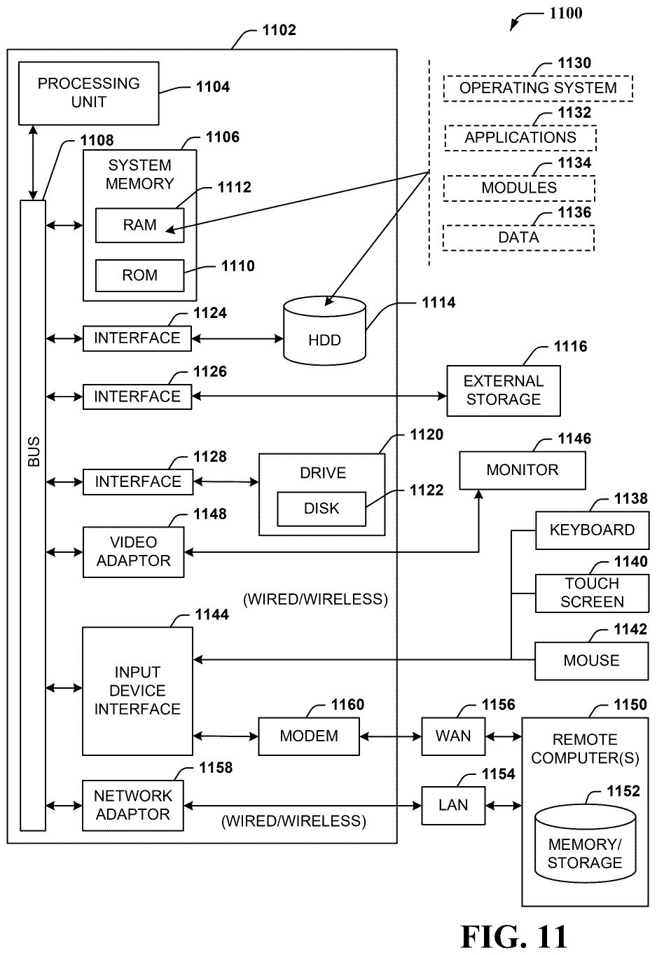

In order to provide additional context for various embodiments described herein, FIG. 11 and the following discussion are intended to provide a brief, general description of a suitable computing environment 1100 in which the various embodiments of the embodiment described herein can be implemented. While the embodiments have been described above in the general context of computer-executable instructions that can run on one or more computers, those skilled in the art will recognize that the embodiments can be also implemented in combination with other program modules or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multi-processor computer systems, minicomputers, mainframe computers, Internet of Things (IOT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 11, the example environment 1100 for implementing various embodiments of the aspects described herein includes a computer 1102, the computer 1102 including a processing unit 1104, a system memory 1106 and a system bus 1108. The system bus 908 couples system components including, but not limited to, the system memory 1106 to the processing unit 1104. The processing unit 1104 can be any of various commercially available processors. Dual microprocessors and other multi-processor architectures can also be employed as the processing unit 1104.

The system bus 1108 can be any of several types of bus structure that can further interconnect to a memory bus (with or without a memory controller), a peripheral bus, and a local bus using any of a variety of commercially available bus architectures. The system memory 1106 includes ROM 1110 and RAM 1112. A basic input/output system (BIOS) can be stored in a non-volatile memory such as ROM, erasable programmable read only memory (EPROM), EEPROM, which BIOS contains the basic routines that help to transfer information between elements within the computer 1102, such as during startup. The RAM 1112 can also include a high-speed RAM such as static RAM for caching data.

The computer 1102 further includes an internal hard disk drive (HDD) 1114 (e.g., EIDE, SATA), one or more external storage devices 1116 (e.g., a magnetic floppy disk drive (FDD) 1116, a memory stick or flash drive reader, a memory card reader, etc.) and a drive 1120, e.g., such as a solid state drive, an optical disk drive, which can read or write from a disk 1122, such as a CD-ROM disc, a DVD, a BD, etc. Alternatively, where a solid state drive is involved, disk 1122 would not be included, unless separate. While the internal HDD 914 is illustrated as located within the computer 1102, the internal HDD 1114 can also be configured for external use in a suitable chassis (not shown). Additionally, while not shown in environment 1100, a solid state drive (SSD) could be used in addition to, or in place of, an HDD 1114. The HDD 1114, external storage device(s) 1116 and drive 1120 can be connected to the system bus 1108 by an HDD interface 1124, an external storage interface 1126 and a drive interface 1128, respectively. The interface 1124 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1102, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1112, including an operating system 1130, one or more application programs 1132, other program modules 1134 and program data 1136. All or portions of the operating system, applications, modules, or data can also be cached in the RAM 1112. The systems and methods described herein can be implemented utilizing various commercially available operating systems or combinations of operating systems.

Computer 1102 can optionally comprise emulation technologies. For example, a hypervisor (not shown) or other intermediary can emulate a hardware environment for operating system 1130, and the emulated hardware can optionally be different from the hardware illustrated in FIG. 11. In such an embodiment, operating system 1130 can comprise one virtual machine (VM) of multiple VMs hosted at computer 1102. Furthermore, operating system 1130 can provide runtime environments, such as the Java runtime environment or the .NET framework, for applications 1132. Runtime environments are consistent execution environments that allow applications 1132 to run on any operating system that includes the runtime environment. Similarly, operating system 1130 can support containers, and applications 1132 can be in the form of containers, which are lightweight, standalone, executable packages of software that include, e.g., code, runtime, system tools, system libraries and settings for an application.

Further, computer 1102 can be enable with a security module, such as a trusted processing module (TPM). For instance with a TPM, boot components hash next in time boot components, and wait for a match of results to secured values, before loading a next boot component. This process can take place at any layer in the code execution stack of computer 1102, e.g., applied at the application execution level or at the operating system (OS) kernel level, thereby enabling security at any level of code execution.

A user can enter commands and information into the computer 1102 through one or more wired/wireless input devices, e.g., a keyboard 1138, a touch screen 1140, and a pointing device, such as a mouse 1142. Other input devices (not shown) can include a microphone, an infrared (IR) remote control, a radio frequency (RF) remote control, or other remote control, a joystick, a virtual reality controller or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1104 through an input device interface 1144 that can be coupled to the system bus 1108, but can be connected by other interfaces, such as a parallel port, an IEEE 1394 serial port, a game port, a USB port, an IR interface, a BLUETOOTH® interface, etc.

A monitor 1146 or other type of display device can be also connected to the system bus 1108 via an interface, such as a video adapter 1148. In addition to the monitor 1146, a computer typically includes other peripheral output devices (not shown), such as speakers, printers, etc.

The computer 1102 can operate in a networked environment using logical connections via wired or wireless communications to one or more remote computers, such as a remote computer(s) 1150. The remote computer(s) 1150 can be a workstation, a server computer, a router, a personal computer, portable computer, microprocessor-based entertainment appliance, a peer device or other common network node, and typically includes many or all of the elements described relative to the computer 1102, although, for purposes of brevity, only a memory/storage device 1152 is illustrated. The logical connections depicted include wired/wireless connectivity to a local area network (LAN) 1154 or larger networks, e.g., a wide area network (WAN) 1156. Such LAN and WAN networking environments are commonplace in offices and companies, and facilitate enterprise-wide computer networks, such as intranets, all of which can connect to a global communications network, e.g., the Internet.

When used in a LAN networking environment, the computer 1102 can be connected to the local network 1154 through a wired or wireless communication network interface or adapter 1158. The adapter 1158 can facilitate wired or wireless communication to the LAN 1154, which can also include a wireless access point (AP) disposed thereon for communicating with the adapter 1158 in a wireless mode.

When used in a WAN networking environment, the computer 1102 can include a modem 1160 or can be connected to a communications server on the WAN 1156 via other means for establishing communications over the WAN 1156, such as by way of the Internet. The modem 1160, which can be internal or external and a wired or wireless device, can be connected to the system bus 1108 via the input device interface 1144. In a networked environment, program modules depicted relative to the computer 1102 or portions thereof, can be stored in the remote memory/storage device 1152. It will be appreciated that the network connections shown are example and other means of establishing a communications link between the computers can be used.

When used in either a LAN or WAN networking environment, the computer 1102 can access cloud storage systems or other network-based storage systems in addition to, or in place of, external storage devices 1116 as described above, such as but not limited to a network virtual machine providing one or more aspects of storage or processing of information. Generally, a connection between the computer 1102 and a cloud storage system can be established over a LAN 1154 or WAN 1156 e.g., by the adapter 1158 or modem 1160, respectively. Upon connecting the computer 1102 to an associated cloud storage system, the external storage interface 1126 can, with the aid of the adapter 1158 or modem 1160, manage storage provided by the cloud storage system as it would other types of external storage. For instance, the external storage interface 1126 can be configured to provide access to cloud storage sources as if those sources were physically connected to the computer 1102.

The computer 1102 can be operable to communicate with any wireless devices or entities operatively disposed in wireless communication, e.g., a printer, scanner, desktop or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

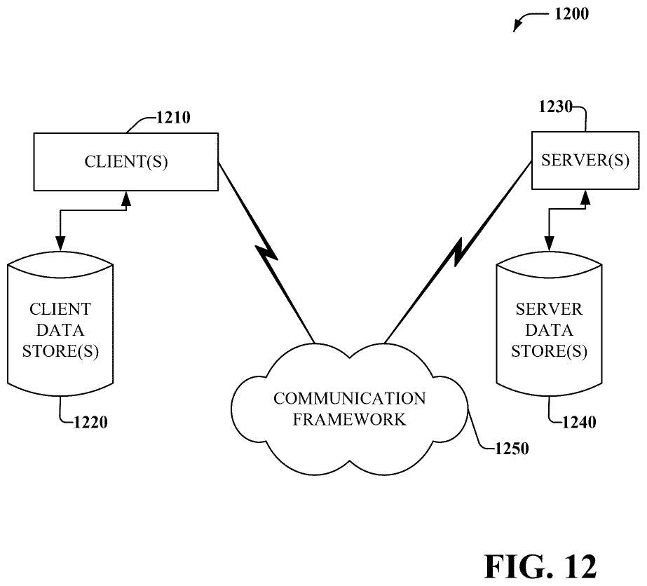

FIG. 12 is a schematic block diagram of a sample computing environment 2000 with which the disclosed subject matter can interact. The sample computing environment 2000 includes one or more client(s) 1210. The client(s) 1210 can be hardware or software (e.g., threads, processes, computing devices). The sample computing environment 1000 also includes one or more server(s) 1230. The server(s) 1230 can also be hardware or software (e.g., threads, processes, computing devices). The servers 1230 can house threads to perform transformations by employing one or more embodiments as described herein, for example. One possible communication between a client 1210 and a server 1230 can be in the form of a data packet adapted to be transmitted between two or more computer processes. The sample computing environment 1200 includes a communication framework 1250 that can be employed to facilitate communications between the client(s) 1210 and the server(s) 1230. The client(s) 1210 are operably connected to one or more client data store(s) 1220 that can be employed to store information local to the client(s) 1210. Similarly, the server(s) 1230 are operably connected to one or more server data store(s) 1240 that can be employed to store information local to the servers 1230.

The present invention may be a system, a method, an apparatus or a computer program product at any possible technical detail level of integration. The computer program product can include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, firewalls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations or block diagrams of methods, apparatus (systems), and computer program products according to embodiments of the invention. It will be understood that each block of the flowchart illustrations or block diagrams, and combinations of blocks in the flowchart illustrations or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowcharts and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can execute from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. As used herein, the term “and/or” is intended to have the same meaning as “or.” Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A system, comprising:
a memory configured to store computer-executable components; and
a processor that executes at least one of the computer-executable components that:
receives Fast Healthcare Interoperability Resources (FHIR) from a FHIR server, the FHIR including unstructured medical report text associated with medical reports; and
extracts unstructured information for the medical reports from the unstructured medical report text by using pre-designed a tree of large language model (LLM) prompts and logic for navigating the tree as a function of prompt responses, wherein given a medical report, a first prompt from the tree is submitted to an LLM service and an answer from the LLM service is used to automatically select a subsequent prompt in the tree without human intervention; and
generates JavaScript Object Notation (JSON) structured data representing extracted entities from the medical reports based on the unstructured information, and integrates the JSON structured data into original FHIR resources as FHIR extension elements.

2. The system of claim 1, further comprising a Natural Language Processing (NLP) system that extracts medical-related entities from the medical reports, wherein the medical-related entities are provided as input to the tree of LLM prompts.

3. The system of claim 1, wherein the at least one of the computer-executable components further:
generates prompts from the tree of LLM prompts, wherein each prompt is associated to medical context of an input and a patient to improve accuracy of the LLM, and wherein the medical comprises at least one of report type, treatment phase, cancer type, or patient state.

4. (canceled)

5. The system of claim 1, wherein the first prompt is designed to extract patient treatment type and clinical phase automatically from the medical report.

6. The system of claim 1, wherein subsequent prompts target specific subsets of extracted entities for refinement or clarification.

7. The system of claim 1, wherein subsequent prompts address ambiguities or conflicts in extracted data and add fine tuning to a core specialty.

8. The system of claim 1, wherein the tree of LLM prompts is fixed, and answers to prompts at a root level of the tree are used to select prompts from one or more lower levels of the tree such that the tree is dynamically expanded.

9. A computer-implemented method, comprising:
receiving, by a system operatively coupled to a processor, Fast Healthcare Interoperability Resources (FHIR) from a FHIR server, the FHIR including unstructured medical report text associated with medical reports; and
extracting, by the system, unstructured information for the medical reports from the unstructured medical report text by using pre-designed a tree of large language model (LLM) prompts and logic for navigating the tree as a function of prompt responses, wherein given a medical report, a first prompt from the tree is submitted to an LLM service and an answer from the LLM service is used to automatically select a subsequent prompt in the tree without human intervention; and
generating, by the system, JavaScript Object Notation (JSON) structured data representing extracted entities from the medical reports based on the unstructured information, and integrates the JSON structured data into original FHIR resources as FHIR extension elements.

10. The computer-implemented method of claim 9, further comprising extracting, by the system, using Natural Language Processing (NLP), medical-related entities from the medical reports, wherein the medical-related entities are provided as input to the tree of LLM prompts.

11. The computer-implemented method of claim 9, further comprising generating, by the system, prompts from the tree of LLM prompts, wherein each prompt is associated to medical context of an input and a patient to improve accuracy of the LLM, and wherein the medical comprises at least one of report type, treatment phase, cancer type, or patient state.

12. (canceled)

13. The computer-implemented method of claim 9, wherein the first prompt is designed to extract patient treatment type and clinical phase automatically from the medical report.

14. The computer-implemented method of claim 9, wherein subsequent prompts target specific subsets of extracted entities for refinement or clarification.

15. The computer-implemented method of claim 9, wherein subsequent prompts address ambiguities or conflicts in extracted data and add fine tuning to a core specialty.

16. The computer-implemented method of claim 9, wherein the tree of LLM prompts is fixed, and answers to prompts at a root level of the tree are used to select prompts from one or more lower levels of the tree such that the tree is dynamically expanded.

17. A computer program product comprising a computer readable storage medium having program instructions embodied therewith, the program instructions executable by a processor to cause the processor to:
receive Fast Healthcare Interoperability Resources (FHIR) from a FHIR server, the FHIR including unstructured medical report text associated with medical reports; and
extract unstructured information for the medical reports from the unstructured medical report text by using pre-designed a tree of large language model (LLM) prompts and logic for navigating the tree as a function of prompt responses, wherein given a medical report, a first prompt from the tree is submitted to an LLM service and an answer from the LLM service is used to automatically select a subsequent prompt in the tree without human intervention; and
generate JavaScript Object Notation (JSON) structured data representing extracted entities from the medical reports based on the unstructured information, and integrates the JSON structured data into original FHIR resources as FHIR extension elements.

18. The computer program product of claim 17, wherein the program instructions are further executable by the processor to cause the processor to:
extract, using Natural Language Processing (NLP), medical-related entities from the medical reports, wherein the medical-related entities are provided as input to the tree of LLM prompts.

19. The computer program product of claim 17, wherein the program instructions are further executable by the processor to cause the processor to:
generate prompts from the tree of LLM prompts, wherein each prompt is associated to medical context of an input and a patient to improve accuracy of the LLM, and wherein the medical comprises at least one of report type, treatment phase, cancer type, or patient state.

20. The computer program product of claim 17, wherein the first prompt is designed to extract patient treatment type and clinical phase automatically from the medical report.

21. The computer program product of claim 17, wherein subsequent prompts target specific subsets of extracted entities for refinement or clarification.

22. The computer program product of claim 17, wherein subsequent prompts address ambiguities or conflicts in extracted data and add fine tuning to a core specialty.
